(19) United States
(12) Patent Application Publication
(10) Pub. No.: US 2016/0177321 A1
(43) Pub. Date: Jun. 23, 2016

(54) RECOMBINANT YEAST CELL AND PREPARATION PROCESS AND USE THEREOF

(71) Applicant: Far Eastern New Century Corporation, Taipei City (TW)

(72) Inventors: Shu-Yin Shih, Taipei City (TW); Der-Ren Hwang, Taipei City (TW)

(21) Appl. No.: 14/928,624

(22) Filed: Oct. 30, 2015

(30) Foreign Application Priority Data
Dec. 22, 2014 (TW) 103144782

(51) Int. Cl. C12N 15/81; C12R 1/865; C12P 7/06

(52) U.S. Cl. CPC C12N 15/81; C12P 7/06; C12R 1/865

(57) ABSTRACT

Disclosed herein is a process for producing a recombinant yeast cell, which includes providing a parent yeast cell that can produce ethanol by consuming a hexose and a pentose, and subjecting the parent yeast cell to a genetic modification treatment. The genetic modification treatment includes deleting, disrupting, or disabling the fps1 gene of the parent yeast cell, introducing a gene encoding xylitol dehydrogenase (XDH) into the genome of the parent yeast cell for over-production of XDH, and deleting, disrupting, or disabling the gpd1 and gpd2 genes of the parent yeast cell in a sequential order.

### CROSS-REFERENCE TO RELATED APPLICATIONS

This application claims priority of Taiwanese Application No. 103144782, filed on Dec. 22, 2014.

### BACKGROUND

1. Field of the Invention

The disclosure relates to a method for producing a recombinant yeast cell, and more particularly to a method for producing a recombinant yeast cell, which involves genetically modifying a parent yeast cell capable of producing ethanol via hexose and pentose consumption so as to enhance the ethanol yield and reduce the by-product production of the same.

2. Background Information

During the conventional fermentation process carried out by microorganisms, xylose is converted to ethanol through one of the following two routes:

(1) oxido-reductase pathway, in which xylose is reduced to xylitol by xylose reductase (XR), xylitol is oxidized to xylulose by xylitol dehydrogenase (XDH), xylulose is phosphorylated to xylulose-5-phosphate by xylulose kinase (XK), and ethanol is produced via pentose phosphate pathway; and

(2) isomerase pathway, in which xylose is converted to xylulose by xylose isomerase, xylulose is phosphorylated to xylulose-5-phosphate by XK, and ethanol is produced via pentose phosphate pathway.

Furthermore, glucose is converted to ethanol through the following steps. Glucose is converted to fructose-1,6-bisphosphate by hexokinase, phosphoglucose isomerase, and phosphofructokinase. Subsequently, fructose-1,6-bisphosphate is converted to glyceraldehyde-3-phosphate and dihydroxyacetone phosphate (DHAP) by fructose-1,6-bisphosphate aldolase. DHAP is converted to glycerol by glycerol-3-phosphate dehydrogenase-1 (GPD1) and glycerol-3-phosphate dehydrogenase-2 (GPD2), and glyceraldehyde-3-phosphate is further converted to ethanol.

However, when microorganisms convert xylose and glucose to ethanol during fermentation, by-products adversely affecting the saccharide utilization rate of microorganisms (e.g. xylitol and glycerol) are produced, thereby reducing the ethanol yield. Therefore, researchers in this field endeavor to develop a method for minimizing the production of by-products during fermentation, so as to enhance the ethanol yield.

Saccharomyces cerevisiae is capable of converting a hexose (e.g. glucose) in a cellulosic hydrolysate to ethanol, and hence has been widely used in industrial fermentation. Nevertheless, a genetically unmodified Saccharomyces cerevisiae strain cannot efficiently utilize a large amount of pentoses (e.g. xylose) in a cellulosic hydrolysate, and would produce glycerol (i.e. a by-product) during ethanol fermentation, thereby being inefficient in producing ethanol. Thus, in recent years, a number of studies aim at improving ethanol production via metabolic engineering. For instance, a gene involved in the xylose metabolic pathway of xylose-fermenting bacteria may be introduced into Saccharomyces cerevisiae, and the xylose-fermenting Saccharomyces cerevisiae thus obtained can effectively co-ferment a pentose and a hexose to achieve a higher ethanol yield (B. Hahn-Hagerdal et al. (2007), Appl. Microbiol. Biotechnol., 74:937-953).

TW 1450963 B (corresponding to US 20140087438 A1 and CN 103695329 A) discloses a xylose-fermenting Saccharomyces cerevisiae strain, which comprises a gene encoding XR (i.e. xr gene), a gene encoding XDH (i.e. xdh gene), and a gene encoding XK (i.e. xk gene), and which is outstanding in terms of xylose consumption rate and ethanol yield. The aforesaid xylose-fermenting Saccharomyces cerevisiae strain has been deposited in Deutsche Sammlung von Mikroorganismen and Zellkulturen (DSMZ) under DSM 25508 and in the Biosource Collection and Research Center (BCRC) of Food Industry Research and Development Institute (FIRDI) under BCRC 920077.

As reported in S. R. Kim et al. (2012), Metabolic Engineering, 14:336-343, each of the two Saccharomyces cerevisiae strains YSX3-tX2 and YSX3-pX2 carries a xr gene, a xdh gene, and a xk gene, and the xdh gene of these two strains is overexpressed. Compared to the Saccharomyces cerevisiae strain YSX3 (which carries a xr gene, a xdh gene, and a xk gene, and in which the xdh gene is not overexpressed), the Saccharomyces cerevisiae strains YSX3-tX2 and YSX3-pX2 are able to utilize a larger amount of xylose during fermentation and have a higher ethanol yield.

Moreover, researcher have attempted to delete the genes involved in glycerol formation and intracellular accumulation of enzymes, and/or the genes encoding plasma membrane transporters, so as to reduce the glycerol yield during fermentation and so as to enhance the ethanol yield. For example, as reported in Zhang A. et al. (2007), Letters in Applied Microbiology, 44:212-217, the fps1 gene encoding a glycerin passage protein in Saccharomyces cerevisiae was deleted to prepare a Δfps1 mutant strain of Saccharomyces cerevisiae. It was found that compared to the Saccharomyces cerevisiae strains subjected to no FPS1 deletion, the Δfps1 mutant strain of Saccharomyces cerevisiae has a lower glycerol yield and a higher ethanol yield.

US 2011/0275130 A1 discloses a recombinant Saccharomyces cerevisiae strain, a Δgpd1Δgpd2 strain expressing the mhpF gene. The recombinant Saccharomyces cerevisiae strain is produced as follows. Saccharomyces cerevisiae strain CEN.PK102-3A is subjected to full-length deletion of the gpd1 and gpd2 genes to obtain a Δgpd1Δgpd2 strain of Saccharomyces cerevisiae, RWB0094. Subsequently, a LEU2 gene encoding β-isopropylmalate dehydrogenase is transformed into Saccharomyces cerevisiae strain RWB0094 to obtain Saccharomyces cerevisiae strain IMZ008. A mhpF gene from Escherichia coli is transformed into Saccharomyces cerevisiae strain IMZ008, so that the aforesaid recombinant Saccharomyces cerevisiae strain is produced. The glycerol and ethanol yields of the aforesaid recombinant Saccharomyces cerevisiae strain in the presence of glucose and acetic acid under an anaerobic condition have been determined. The experimental results indicate that compared to Saccharomyces cerevisiae strain IME076 which carries gpd1 and gpd2 genes, the aforesaid recombinant Saccharomyces cerevisiae strain leads to no glycerol formation and has a higher ethanol yield. However, when glucose serves as the only carbon source, the aforesaid recombinant Saccharomyces cerevisiae strain cannot grow under an anaerobic condition.

US 2011/0250664 A1 discloses a genetically-altered Saccharomyces cerevisiae strain, which is produced by subjecting Saccharomyces cerevisiae strain YC-DM to full-length deletion of the fps1 and gpd2 genes, truncation of the promoter sequence of the gpd1 gene, and overexpression of the gene of glutamate synthase 1 (GLT1). The aforesaid genetically-altered Saccharomyces cerevisiae strain was inoculated into corn mash so as to conduct fermentation, and the ethanol and glycerol yields were determined. The experimental results indicate that compared to commercially available Saccharomyces cerevisiae strains (BIOFERM XR and ETHANOL RED®), the aforesaid genetically-altered Saccharomyces cerevisiae strain produces less glycerol and has a higher ethanol yield.

As described in Hubmann G. et al. (2011), Applied and Environmental Microbiology, 77:5857-5867, a Δgpd1 deletion strain of Saccharomyces cerevisiae, a Δgpd2 deletion strain of Saccharomyces cerevisiae, and a Δgpd1 Δgpd2 double deletion stain of Saccharomyces cerevisiae were obtained by subjecting the gpd1 gene of a wild-type strain of Saccharomyces cerevisiae to disruption and/or subjecting the promoter of the gpd2 gene of such strain to replacement. The ethanol and glycerol production regarding the aforesaid deletion strains of Saccharomyces cerevisiae were evaluated under quasi-anaerobic fermentation conditions. The experimental results reveal that compared to the wild-type strain of Saccharomyces cerevisiae, the aforesaid deletion strains of Saccharomyces cerevisiae produce less glycerol and have a higher ethanol yield. In particular, the Δgpd1Δgpd2 double deletion stain of Saccharomyces cerevisiae leads to no glycerol formation. However, the Δgpd1Δgpd2 double deletion strain of Saccharomyces cerevisiae is unable to completely ferment sugars under anaerobic conditions.

In spite of the conventional yeast strains, there is still a need to develop a recombinant yeast cell that can consume a biomass containing a hexose (e.g. glucose) and a pentose (e.g. xylose), and that has a high ethanol conversion rate and a satisfactorily low yield of a by-product.

### SUMMARY

Accordingly, in a first aspect, the present disclosure provides a process for producing a recombinant yeast cell, which comprises:

providing a parent yeast cell having a genome that includes genes enabling the parent yeast cell to produce ethanol by consuming a hexose and a pentose, a gene encoding xylose reductase (XR), a first gene encoding xylitol dehydrogenase (XDH), and a gene encoding xylulose kinase (XK); and

subjecting the parent yeast cell to a genetic modification treatment, which includes deleting, disrupting, or disabling the fps1 gene in the genome of the parent yeast cell, introducing a second gene encoding XDH into the genome of the parent yeast cell for over-production of XDH, and deleting, disrupting, or disabling the gpd1 and gpd2 genes in the genome of the parent yeast cell in a sequential order.

In a second aspect, the present disclosure provides a recombinant yeast cell which is produced by a process as described above.

In a third aspect, the present disclosure provides a method for producing ethanol from a biomass containing a hexose and/or a pentose, which comprises subjecting the biomass to fermentation with a recombinant yeast cell able to produce ethanol by consuming a hexose and a pentose. The recombinant yeast cell is produced by a process as described above.

### BRIEF DESCRIPTION OF THE DRAWINGS

The above and other objects, features and advantages of the present disclosure will become apparent with reference to the following detailed description and the exemplary embodiments taken in conjunction with the accompanying drawings, in which:

FIG. 1 is a schematic diagram showing a process of knocking out a target gene of a parent yeast cell via a gene knockout technique;

FIG. 2 is a schematic diagram of a yTA-FPS-loxpKanMX recombinant vector;

FIG. 3 is a schematic diagram of a yTA-GPD1-loxpKanMX recombinant vector;

FIG. 4 is a schematic diagram of a yTA-GPD2-loxpKanMX recombinant vector;

FIG. 5 is a schematic diagram of a puc-d-loxpKanMX-ENO1-psXDH recombinant vector; and

FIG. 6 is a schematic diagram of a pFENC-Cre recombinant vector.

### DETAILED DESCRIPTION

Unless defined otherwise, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described. For the sake of clarity, the following definitions are used herein.

As used herein, the term “delete” refers to partial or entire removal of a coding region of a gene.

As used herein, the term “disrupt” refers to removal, insertion, or mutation of a nucleotide of a gene.

As used herein, the term “disable” refers to inactivating a gene or the protein encoded by the gene so as to force the gene or protein to lose its activity or function.

As used herein, the terms “over-production” and “over-expression” can be interchangeably used, and refer to an expression level or a production level of a protein or metabolite in a cell, which is higher than the level of the protein or metabolite needed by the cell, and which might lead to the accumulation and storage of the protein or metabolite in the cell.

As used herein, the terms “parent yeast cell” and “yeast mother strain” can be interchangeably used, and refer to a yeast cell used to conduct one or more genetic modification treatments. The parent yeast cells suitable for the present disclosure may be non-transformed cells or cells transformed with at least one recombinant nucleic acid.

The parent yeast cells suitable for the present disclosure include, but are not limited to, cells of the following species: Saccharomyces spp., Pichia spp., Candida spp., and Pachysolen spp. The parent yeast cells suitable for the present disclosure may be cells of xylose-utilizing Saccharomyces cerevisiae, cells of Pichia stipitis, cells of Candida shehatae, or cells of Pachysolen tannophilus. In one exemplary embodiment of the present disclosure, the parent yeast cell to be used is a cell of xylose-utilizing Saccharomyces cerevisiae.

As used herein, the terms “xylose-utilizing Saccharomyces cerevisiae” and “xylose-fermenting Saccharomyces cerevisiae” can be interchangeably used, and are intended to cover all the Saccharomyces cerevisiae strains having xylose fermentation ability.

The present disclosure provides a process for producing a recombinant yeast cell, which comprises:

providing a parent yeast cell having a genome that includes genes enabling the parent yeast cell to produce ethanol by consuming a hexose and a pentose, a gene encoding xylose reductase (XR), a first gene encoding xylitol dehydrogenase (XDH), and a gene encoding xylulose kinase (XK); and

subjecting the parent yeast cell to a genetic modification treatment, which includes deleting, disrupting, or disabling the fps1 gene in the genome of the parent yeast cell, introducing a second gene encoding XDH into the genome of the parent yeast cell for over-production of XDH, and deleting, disrupting, or disabling the gpd1 and gpd2 genes in the genome of the parent yeast cell in a sequential order.

According to the present disclosure, the genetic modification treatment is conducted using the following sequential steps:

deleting, disrupting, or disabling the fps1 gene in the genome of the parent yeast cell;

introducing the second gene encoding XDH into the genome of the parent yeast cell for over-production of XDH;

deleting, disrupting, or disabling the gpd1 gene in the genome of the parent yeast cell; and

deleting, disrupting, or disabling the gpd2 gene in the genome of the parent yeast cell.

According to the present disclosure, when the parent yeast cell is a cell of xylose-utilizing Saccharomyces cerevisiae, the gene encoding XR and the first and second genes encoding XDH in the genome of the parent yeast cell are exogenous, are expressable in the parent yeast cell, and are derived from the genome of any one of the following yeasts: Pichia stipitis, Candida shehatae, and Pachysolen tannophilus. In an exemplary embodiment of the present disclosure, the gene encoding XR and the first and second genes encoding XDH are derived from the genome of Pichia stipitis.

According to the present disclosure, the genome of the parent yeast cell includes a fps1 gene encoding a glycerin passage protein, a gpd1 gene encoding glycerol-3-phosphate dehydrogenase-1 (GPD1), and a gpd2 gene encoding glycerol-3-phosphate dehydrogenase-2 (GPD2).

In an exemplary embodiment of the present disclosure, the parent yeast cell is a cell of the Saccharomyces cerevisiae strain deposited under the accession number BCRC 920077 or DSM 25508.

In an exemplary embodiment of the present disclosure, during the genetic modification treatment, the full-length nucleic acid sequence of the fps1 gene, at least 80% of the nucleic acid sequence of the gpd1 gene, and the full-length nucleic acid sequence of the gpd2 gene in the genome of the parent yeast cell are deleted using a gene knockout technique well-known to and commonly used by one skilled in the art, e.g. the techniques described in Zhang A. et al. (2007), supra, Hubmann G. et al. (2011), supra, US 2011/0275130 A1, and US 2011/0250664 A1.

According to the present disclosure, the hexose that can be consumed by the parent yeast cell is selected from the group consisting of glucose, galactose, fructose, mannose, and combinations thereof. According to the present disclosure, the pentose that can be consumed by the parent yeast cell is selected from the group consisting of xylose, arabinose, and a combination thereof.

In an exemplary embodiment of the present disclosure, the parent yeast cell can produce ethanol by consuming glucose and xylose.

The present disclosure also provides a recombinant yeast cell which is produced by a process as described above.

In an exemplary embodiment of the present disclosure, the recombinant yeast cell is a cell of the yeast strain deposited under the accession number BCRC 920086 or DSM 28105.

The present disclosure also provides a method for producing ethanol from a biomass containing a hexose and/or a pentose, which comprises subjecting the biomass to fermentation with a recombinant yeast cell able to produce ethanol by consuming a hexose and a pentose. The recombinant yeast cell is produced by a process as described above.

According to the present disclosure, the biomass is a mixed sugar liquor containing a hexose and/or a pentose. In an exemplary embodiment of the present disclosure, the biomass is a mixed sugar liquor containing glucose and xylose.

According to the present disclosure, the biomass is a plant biomass containing a hexose and/or a pentose. The biomass may be a cellulosic hydrolysate obtained from a plant material and containing glucose and xylose. In an exemplary embodiment of the present disclosure, the biomass is a cellulosic hydrolysate of rice straw, which is produced via saccharification of rice straw.

In an exemplary embodiment of the present disclosure, when a mixed sugar liquor containing glucose and xylose is used as the biomass for fermentation, the recombinant yeast cell of the present disclosure has an ethanol yield of at least about 0.75 g/g. In another exemplary embodiment of the present disclosure, when a mixed sugar liquor containing glucose and xylose is used as the biomass for fermentation, the recombinant yeast cell of the present disclosure has an ethanol yield of at least about 0.8 g/g. In still another exemplary embodiment of the present disclosure, when a mixed sugar liquor containing glucose and xylose is used as the biomass for fermentation, the recombinant yeast cell of the present disclosure has an ethanol yield of about 0.833 g/g.

In an exemplary embodiment of the present disclosure, when a cellulosic hydrolysate of rice straw is used as the biomass for fermentation, the recombinant yeast cell of the present disclosure has an ethanol yield of at least about 0.9 g/g. In another exemplary embodiment of the present disclosure, when a cellulosic hydrolysate of rice straw is used as the biomass for fermentation, the recombinant yeast cell of the present disclosure has an ethanol yield of about 0.909 g/g.

The present disclosure will be further described by way of the following examples. However, it should be understood that the following examples are solely intended for the purpose of illustration and should not be construed as limiting the present disclosure in practice.

### EXAMPLES

### Experimental Materials

1. The restriction enzymes used in the examples (Thermo Scientific FastDigest enzymes) were all purchased from Level Biotechnology Inc.

2. The primers for polymerase chain reaction (PCR) as used in the examples were synthesized by Mission Biotech Co., Ltd.

3. The following experimental materials were purchased from Yeastern Biotech: yT&A cloning vector kit (Cat. No. FYC001) and UniversAll™ tissue extraction buffer (Cat. No. FYU002). yT&A cloning vector kit comprises a yT&A cloning vector (2728 bp) with an ampicillin resistance gene and a β-galactosidase coding gene.

4. The vectors used in the examples are as follows:
(1) pUC19 vector (2686 bp), which has an ampicillin resistance gene, a β-galactosidase coding gene and an origin of replication (ori), was purchased from Level Biotechnology Inc. (Cat. No. SD0061).
(2) pFA6a-link-yEGFP-Kan vector (4894 bp), which has a KanMX resistance gene (i.e. nucleotide residues 962 to 2392 of pFA6a-link-yEGFP-Kan vector), was purchased from European Saccharomyces Cerevisiae Archive For Functional Analysis (EUROSCARF).
(3) pFA6-hphMX6 vector (4157 bp), which has a hygromycin resistance gene (i.e. nucleotide residues 71 to 1720 of pFA6-hphMX6 vector), was purchased from EUROSCARF.
(4) pYD1 vector (5009 bp), which has a GAL1 promoter (i.e. nucleotide residues 1 to 451 of pYD1 vector), was purchased from Invitrogen (Cat. No. V835-01).
(5) pSos vector (11,259 bp), which has a 2u on fragment (i.e. nucleotide residues 7901 to 8750 of pSos vector), was purchased from Agilent Technologies (Cat. No. 217438).

5. The yeast strains used in the examples are as follows:
(1) A Saccharomyces cerevisiae strain, which has been deposited in the Biosource Collection and Research Center (BCRC) under an accession number BCRC 920077 and in the Deutsche Sammlung von Mikroorganismen and Zellkulturen GmbH (DSMZ) under an accession number DSM 25508, was purchased from the BCRC.
(2) A Pichia stipitis strain, which has been deposited in the BCRC under an accession number BCRC 21775 and in the American Type Culture Collection (ATCC) under an accession number ATCC 58376, was purchased from the BCRC.

6. The following control standards for high performance liquid chromatography (HPLC) were all purchased from Sigma: glucose (1.25 to 24 mg/mL), xylose (1.25 to 24 mg/mL), xylitol (0.25 to 6 mg/mL), glycerol (0.375 to 8 mg/mL) and ethanol (0.93 to 20 mg/mL).

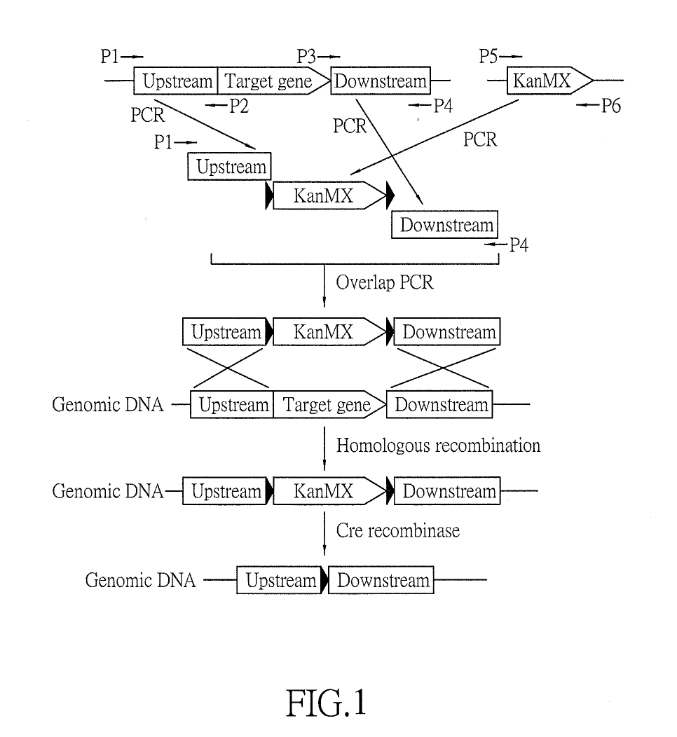

General Experimental Procedures:
1. Unless otherwise indicated, the experimental procedures below (including DNA cloning procedures) were performed by virtue of techniques well known to those skilled in the art or in accordance with the manufacturer's instructions.
2. A gene knockout was carried out to knock out a target gene in a parent yeast cell. The flow chart for the aforesaid gene knockout is shown in FIG. 1, in which “target gene” represents a target gene to be knocked out; “upstream” and “downstream” respectively represent an upstream fragment and a downstream fragment of the target gene; “KanMX” represents a KanMX resistance gene; “P1” and “P2” represent primers for amplifying the upstream fragment of the target gene; “P3” and “P4” represent primers for amplifying the downstream fragment of the target gene; “P5” and “P6” represent primers for amplifying the KanMX resistance gene (the thus amplified PCR product is a loxp-KanMX-loxp fragment containing a KanMX resistance gene, and each loxp sequence of the loxp-KanMX-loxp fragment is represented by a black triangle); “PCR” represents polymerase chain reaction; and “overlap PCR” represents overlap polymerase chain reaction.
3. Preparation of yeast cultures

The cultures of Saccharomyces cerevisiae DSM 25508, Saccharomyces cerevisiae transformants, and Pichia stipitis ATCC 58376 for genomic DNA extraction as used in the examples were prepared as follows. Each yeast strain was inoculated into a flask containing 10 mL of a YPD medium supplemented with 1% yeast extract, 2% peptone and 2% glucose, followed by cultivation in a shaking incubator (30° C., 150-200 rpm) for 24 hours.
4. PCR

PCR or overlap PCR was conducted using a KOD DNA polymerase (Merck Taiwan) in accordance with the manufacturer's instructions.
5. Transformation

A desired DNA fragment was transformed into a target yeast by virtue of electroporation (1,500 V, 25 μF, and 200Ω). Subsequently, screening was conducted using a YPD solid medium containing an appropriate antibiotic concentration (300 μg/mL G418 or 500 μg/mL Hygromycin) so as to obtain a yeast transformant.
6. Treatment after gene knockout

A sporulation process was conducted after Saccharomyces cerevisiae was subjected to the transformation process for gene knockout. The sporulation process is described in detail as follows. The Saccharomyces cerevisiae transformant was inoculated into 10 mL of a YPD liquid medium, followed by conducting cultivation in a shaking incubator (30° C., 200 rpm) until an OD₆₀₀ value of 1 was reached. Centrifugation was performed. The resultant pellet was collected, followed by washing three times with sterilized water. The yeasts thus obtained were inoculated into 50 mL of a YPK medium containing 20 g/L yeast extract, 10 g/L peptone and 10 g/L potassium acetate, followed by cultivation at 30° C. and 200 rpm overnight. Centrifugation was performed, and the pellet was collected, followed by washing three times with sterilized water. The yeasts thus obtained were inoculated into 50 mL of a sporulation medium containing 10 g/L potassium acetate, 1.0 g/L yeast extract, 0.5 g/L glucose, 0.05 g/L adenosine, 0.05 g/L uridine, 0.1 g/L tryptophan, 0.1 g/L leucine and 0.1 g/L histidine. Cultivation was performed at 30° C. and 200 rpm for 6 days so as to form haploid Saccharomyces cerevisiae transformants.

After the aforesaid sporulation process was accomplished, an appropriate amount of the resultant yeast solution of the haploid Saccharomyces cerevisiae transformants was diluted. The diluted yeast solution was spread onto a YPD solid medium containing 300 μg/mL G418, followed by conducting cultivation so as to obtain diploid Saccharomyces cerevisiae transformants. Afterward, a PCR process was conducted to confirm whether the target gene had been knocked out.
7. Removal of KanMX resistance gene

The process of removing the KanMX resistance gene is described in detail as follows. The recombinant vector pFENC-Cre constructed in Example 3 was transformed into a target yeast transformant according to the method set forth in section 5 of “General experimental procedures”, and the resultant yeast transformant was cultivated in a YPD solid medium containing 500 μg/mL hygromycin for 48 hours so as to obtain a pFENC-Cre containing yeast transformant. The pFENC-Cre containing yeast transformant thus obtained was inoculated into a galactose induction solution containing 20 g/L galactose, 1.74 g/L yeast nitrogen base and 5 g/L ammonium sulphate, followed by cultivation at 30° C. and 200 rpm for 48 hours. A portion of the resultant yeasts were cultivated in a YPD solid medium for 24 hours. A single colony was selected, and three portions of the selected colony were respectively inoculated into a YPD solid medium, a YPD solid medium supplemented with 300 μg/mL G418, and a YPD solid medium supplemented with 500 μg/mL hygromycin. Subsequently, cultivation was performed for 24 hours. Finally, the strain, which can grow only in a YPD solid medium without G418 or hygromycin, was used for the subsequent experiments.
8. HPLC analysis

The components and concentration (g/L) thereof in a test sample were determined using a HPLC instrument (DIONEX Ultimate 3000) equipped with a refractive index detector (RI detector). The column used is an Aminex HPX-87H column (BioRad), and the operating conditions are as follows: column temperature: 65° C.; mobile phase: 5 mM sulfuric acid in water; flow rate: 0.6 mL/minute; a sample injection volume of 20 μL; and RI detector temperature: 45° C.

### Example 1

### Construction of yTA-FPS-loxpKanMX, yTA-GPD1-loxpKanMX, and yTA-GPD2-loxpKanMX Recombinant Vectors

In order to knock out the target genes (i.e. fps1 gene, gpd1 gene, and gpd2 gene) in the genome of Saccharomyces cerevisiae DSM 25508, yTA-FPS-loxpKanMX, yTA-GPD1-loxpKanMX, and yTA-GPD2-loxpKanMX recombinant vectors were constructed as follows.

### A. Cloning of Upstream and Downstream Fragments of Target Genes

The six primer pairs shown in Table 1 were used to clone the following DNA fragments of Saccharomyces cerevisiae DSM 25508: an upstream fragment of fps1 gene (hereinafter referred to as “Fps1-F fragment” and corresponding to nucleotide residues 49513 to 49703 of the nucleotide sequence having NCBI Accession No. BK006945.2), a downstream fragment of fps1 gene (hereinafter referred to as “Fps1-R fragment” and corresponding to nucleotide residues 52031 to 52180 of the nucleotide sequence having NCBI Accession No. BK006945.2), an upstream fragment of gpd1 gene (hereinafter referred to as “Gpd1-F fragment” and corresponding to nucleotide residues 411680 to 411900 of the nucleotide sequence having NCBI Accession No. BK006938.2), a downstream fragment of gpd1 gene (hereinafter referred to as “Gpd1-R fragment” and corresponding to nucleotide residues 412863 to 413086 of the nucleotide sequence having NCBI Accession No. BK006938.2), an upstream fragment of gpd2 gene (hereinafter referred to as “Gpd2-F fragment” and corresponding to nucleotide residues 216725 to 216880 of the nucleotide sequence having NCBI Accession No. BK006948.2), and a downstream fragment of gpd2 gene (hereinafter referred to as “Gpd2-R fragment” and corresponding to nucleotide residues 218513 to 218650 of the nucleotide sequence having NCBI Accession No. BK006948.2).

TABLE 1

| | | Size of |
|---|---|---|
| | | PCR |
| DNA | Nucleotide | product |
| fragment | sequence (5′→3′) | (bp) |
| Fps1-F | Forward primer | 224 |
| fragment | FPS1-F-BglII-F | |
| | agactcagatctctcgtc | |
| | ctaaagtcaattagttc | |
| | BglII | |
| | (SEQ ID NO: 1) | |
| | Reverse primer | |
| | FPS1-F-LoKa-NdeI-R | |
| | cattatacgaagttatcatatgg | |
| | ccttctcactatgagatctgat | |
| | NdeI | |
| | (SEQ ID NO: 2) | |
| Fps1-R | Forward primer | 183 |
| fragment | FPS1-R-LoKa-SacI-F | |
| | gctatacgaagttatgagctc | |
| | tatatatatttacatagatg | |
| | SacI | |
| | (SEQ ID NO: 3) | |
| | Reverse primer | |
| | FPS1-R-SalI-R | |
| | gtcgacgtcgactatag | |
| | taggtgaccaggctgag | |
| | SalI | |
| | (SEQ ID NO: 4) | |
| Gpd1-F | Forward primer | 255 |
| fragment | GPD1-F-XhoI-F | |
| | ctcgagctcgagctacca | |
| | tgagtgaaactgttacg | |
| | XhoI | |
| | (SEQ ID NO: 5) | |
| | Reverse primer | |
| | GPD1-F-LoKa-NdeI-R | |
| | cattatacgaagttatcatatgc | |
| | agaagaggaacttctctttctac | |
| | NdeI | |
| | (SEQ ID NO: 6) | |
| Gpd1-R | Forward primer | 257 |
| fragment | GPD1-R-LoKa-SacI-F | |
| | gctatacgaagttatgagctcgt | |
| | tcacgaatggttggaaacatgtg | |
| | SacI | |
| | (SEQ ID NO: 7) | |
| | Reverse primer | |
| | GPD1-R-SalI-R | |
| | gtcgacgtcgacgacataa | |
| | tgctaatttatgaatatg | |
| | SalI | |
| | (SEQ ID NO: 8) | |
| Gpd2-F | Forward primer | 190 |
| fragment | GPD2-F-BglII-F | |
| | agactcagatctgagtat | |
| | gctcgaaacaataagac | |
| | BglII | |
| | (SEQ ID NO: 9) | |
| | Reverse primer | |
| | GPD2-F-LoKa-NdeI-R | |
| | cattatacgaagttatcatatg | |
| | gagaagagctgctgaacaatag | |
| | NdeI | |
| | (SEQ ID NO: 10) | |
| Gpd2-R | Forward primer | 171 |
| fragment | GPD2-R-LoKa-SacI-F | |
| | gctatacgaagttatgagctcc | |
| | attatacacaagttctacaac | |
| | SacI | |
| | (SEQ ID NO: 11) | |
| | Reverse primer | |
| | GPD2-R-SalI-R | |
| | gtcgacgtcgaccgataa | |
| | tagcgtgtataatggtag | |
| | SalI | |
| | (SEQ ID NO: 12) | |

Note: The underlined nucleotides represent the recognition site of the restriction enzyme indicated therebelow.

A suitable amount of a culture of Saccharomyces cerevisiae DSM 25508 was subjected to genomic DNA extraction using UniversAll™ tissue extract buffer. Subsequently, a PCR process was conducted with the extracted genomic DNA serving as templates and the primer pairs shown in Table 1 according to the method described in section 4 of “General Experimental Procedures”. Consequently, PCR products respectively containing the aforesaid six DNA fragments were obtained.

### B. Cloning of Loxp-KanMX-Loxp Fragment

A Loxp-KanMX-Loxp fragment was prepared substantially according to the procedures set forth in Brian Sauer (1987), Mol. Cell. Biol., 7:2087-2096. Briefly, a PCR process was conducted with a pFA6a-link-yEGFP-Kan vector serving as a template and a primer pair designed based on the KanMX resistance gene of the aforesaid vector (as shown below) according to the method described in section 4 of “General Experimental Procedures”, so that a PCR product (1544 bp) containing a Loxp-KanMX-Loxp fragment was obtained.

```
Forward primer loxpKanMX-NdeI-F
(SEQ ID NO: 13)
5′-catatgcatatgataacttcgtataatgtatgctatacgaagttat
                    NdeI
ttcgagaactgctctgtttagcttgcctcg-3′
Reverse primer loxpKanMX-SacI-R
(SEQ ID NO: 14)
5′-gagttcgagctcataacttcgtatagcatacattatacgaagttat
                    SacI
gtatcgacactggatggcggcgttagtat-3′
Note:
The underlined nucleotides represent the recognition site of the restriction enzyme indicated therebelow, and the italic nucleotides represent the loxp sequence.
```

### C. Preparation of Spliced Fragments

The Fps1-F fragment, Loxp-KanMX-Loxp fragment, and Fps1-R fragment obtained from the preceding sections of this example were used to prepare a dF spliced fragment (hereinafter referred to as “dF fragment”). Briefly, an overlap PCR process was performed using a template mixture (containing a Fps1-F fragment, a Loxp-KanMX-Loxp fragment and a Fps1-R fragment in a ratio of 2:1:2), as well as the forward primer FPS1-F-BglII-F (SEQ ID NO:1) and the reverse primer FPS1-R-SalI-R (SEQ ID NO:4) shown in Table 1, according to the method described in section 4 of “General Experimental Procedures”, so that a dF fragment (about 1896 bp) containing the three fragments in the order mentioned above was obtained.

A dG1 spliced fragment (hereinafter referred to as “dG1 fragment”; 2001 bp) was prepared substantially according to the procedures for preparing a dF fragment as described above, except that a template mixture containing a Gpd1-F fragment, a Loxp-KanMX-Loxp fragment and a Gpd1-R fragment was used, and that the forward primer GPD1-F-XhoI-F (SEQ ID NO:5) and the reverse primer GPD1-R-SalI-R (SEQ ID NO:8) shown in Table 1 served as primers.

A dG2 spliced fragment (hereinafter referred to as “dG2 fragment”; 1850 bp) was prepared substantially according to the procedures for preparing a dF fragment as described above, except that a template mixture containing a Gpd2-F fragment, a Loxp-KanMX-Loxp fragment and a Gpd2-R fragment was used, and that the forward primer GPD2-F-BglII-F (SEQ ID NO:9) and the reverse primer GPD2-R-SalI-R (SEQ ID NO:12) shown in Table 1 served as primers.

### D. Construction of Recombinant Vectors

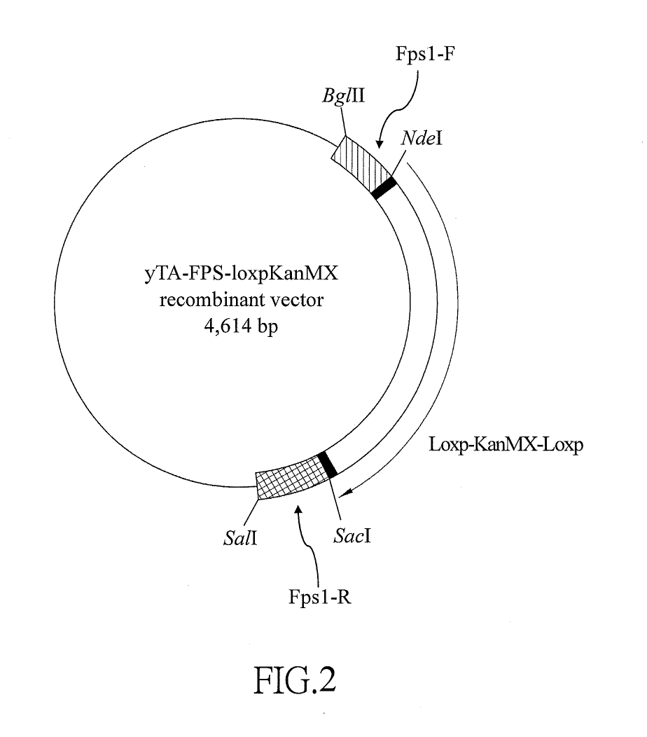

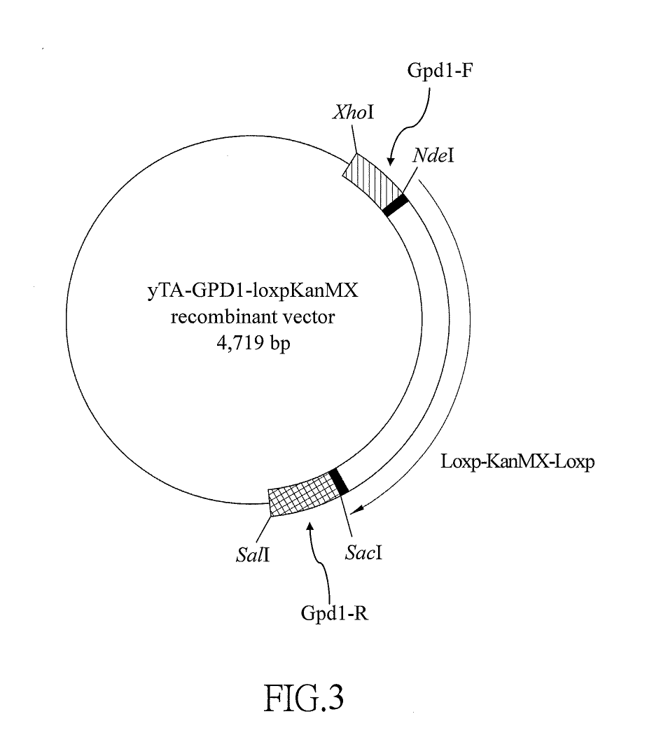

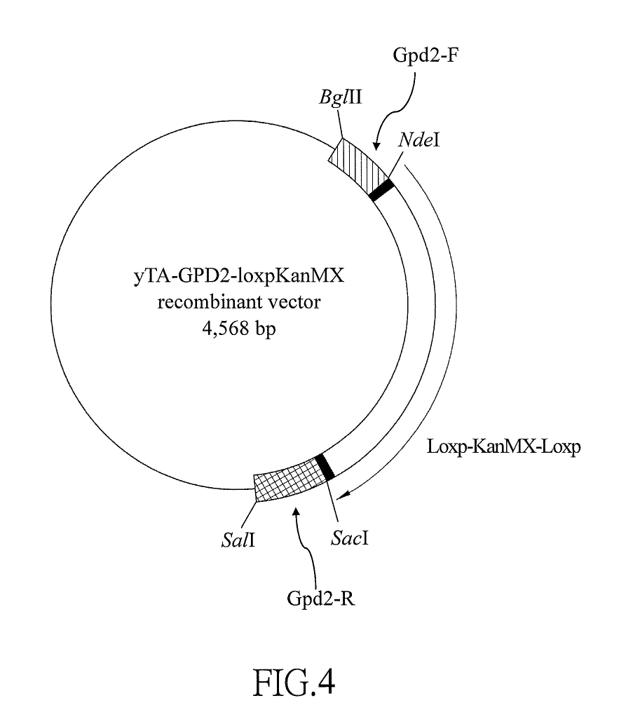

Each of the dF fragment, dG1 fragment and dG2 fragment obtained from section C of this example was cloned into a yT&A cloning vector (2728 bp) using suitable restriction enzymes (e.g., BglII/SalI or XhoI/SalI). Therefore, a yTA-FPS-loxpKanMX recombinant vector (4614 bp) having a structure shown in FIG. 2, a yTA-GPD1-loxpKanMX recombinant vector (4719 bp) having a structure shown in FIG. 3, and a yTA-GPD2-loxpKanMX recombinant vector (4568 bp) having a structure shown in FIG. 4 were obtained.

### Example 2

### Construction of puc-d-loxpKanMX-ENO1-psXDH Recombinant Vector Containing Xdh Gene of Pichia stipitis

In order to overexpress the xdh gene of Pichia stipitis (hereinafter referred to as psXDH gene) in Saccharomyces cerevisiae DSM 25508, a puc-d-loxpKanMX-ENO1-psXDH recombinant vector carrying a Delta sequence, a Loxp-KanMX-Loxp fragment, an ENO1 promoter, a psXDH gene, and an ENO1 terminator were constructed as follows.

A. Cloning of psXDH Gene, Delta Sequence, ENO1 Promoter, and ENO1 Terminator

The five primer pairs shown in Table 2 were used to clone the psXDH gene of Pichia stipitis ATCC 58376 (corresponding to nucleotide residues 51 to 1142 of the nucleotide sequence having NCBI Accession No. XM_001386945.1) and the following DNA fragments of Saccharomyces cerevisiae DSM 25508: the Delta sequence contained in retrotransposons (corresponding to nucleotide residues 96941 to 96614 of the nucleotide sequence having NCBI Accession No. BK006947.3), the ENO1 promoter (corresponding to nucleotide residues 1000330 to 1000926 of the nucleotide sequence having NCBI Accession No. BK006941.2), and the ENO1 terminator (corresponding to nucleotide residues 1002241 to 1002725 of the nucleotide sequence having NCBI Accession No. BK006941.2). The differences between the primer pairs Delta-BglII-F/Delta-NdeI-R and Delta-BamHI-F/Delta-SalI-R both designed based on the Delta sequence reside in the sequences of the recognition sites for restriction enzymes.

TABLE 2

| | | | Size of |
|---|---|---|---|
| | | | PCR |
| | Nucleotide sequence | PCR | product |
| Primer | (5′→3′) | Template | (bp) |
| Forward | AvrII | The genomic | 1118 |
| primer | cctaggcctaggatgactg | DNA of Pichia | |
| psXDH- | ctaacccttccttg | stipitis ATCC | |
| AvrII-F | (SEQ ID NO: 15) | 58376 | |
| Reverse | NotI | | |
| primer | gcggccgcggccgttactc | | |
| psXDH- | agggccgtcaatgag | | |
| NotI-R | (SEQ ID NO: 16) | | |
| Forward | BglII | The genomic | 352 |
| primer | ctgtcaagatcttgttgga | DNA of | |
| Delta- | atagaaatc | Saccharomyces | |
| BglII-F | (SEQ ID NO: 17) | cerevisiae | |
| Reverse | NdeI | DSM 25508 | |
| primer | ctgtcacatatgaaatggg | | |
| Delta- | tgaatgttgag | | |
| NdeI-R | (SEQ ID NO: 18) | | |
| Forward | BamHI | The genomic | 352 |
| primer | ctgtcaggatcctgttgga | DNA of | |
| Delta- | atagaaatc | Saccharomyces | |
| BamHI-F | (SEQ ID NO: 19) | cerevisiae | |
| Reverse | SalI | DSM 25508 | |
| primer | ctgtcagtcgacaaatggg | | |
| Delta- | tgaatgttgag | | |
| SalI-R | (SEQ ID NO: 20) | | |
| Forward | SacI | The genomic | 621 |
| primer | gagctcgagctccggaacc | DNA of | |
| ENO1p- | gccagatattc | Saccharomyces | |
| SacI-F | (SEQ ID NO: 21) | cerevisiae | |
| Reverse | AvrII | DSM 25508 | |
| primer | cctaggcctaggtttgatt | | |
| ENO1p- | tagtgtttgtgtgttg | | |
| AvrII-R | (SEQ ID NO: 22) | | |
| Forward | NotI | The genomic | 517 |
| primer | gcggccgcggccgcgaatt | DNA of | |
| ENO1t- | cagtttttgattaagcctt | Saccharomyces | |
| NotI-F | ctag | cerevisiae | |
| | (SEQ ID NO: 23) | DSM 25508 | |
| Reverse | BamHI | | |
| primer | ggatccggatcccttcatt | | |
| ENO1t- | gagcttagaacccttttg | | |
| BamHI-R | (SEQ ID NO: 24) | | |

Note:
The underlined nucleotides represent the recognition site of the restriction enzyme indicated thereabove.

A PCR process was conducted with the genomic DNA of Pichia stipitis ATCC 58376 or Saccharomyces cerevisiae DSM 25508 serving as templates and the primer pairs shown in Table 2 according to the method described in section 4 of “General Experimental Procedures”. Consequently, a PCR product containing a psXDH gene (hereinafter referred to as “PCR product A1”; 1118 bp), a PCR product containing a Delta sequence and BglII/NdeI recognition sites (hereinafter referred to as “PCR product A2”; 352 bp), a PCR product containing a Delta sequence and BamHI/SalI recognition sites (hereinafter referred to as “PCR product A3”; 352 bp), a PCR product containing an ENO1 promoter (hereinafter referred to as “PCR product A4”; 621 bp), and a PCR product containing an ENO1 terminator (hereinafter referred to as “PCR product A5”; 517 bp) were obtained.

### B. Construction of Puc-d-loxpKanMX-ENO1-psXDH Recombinant Vector

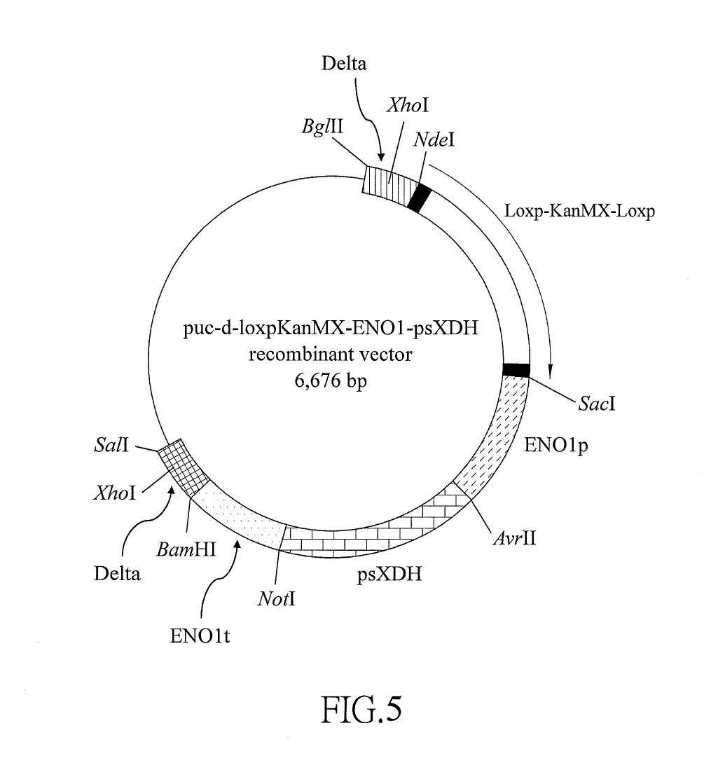

PCR products A1 to A5 obtained from section A of this example and the Loxp-KanMX-Loxp fragment obtained from section B of Example 1 were all cloned into a pUC19 vector (2686 bp) using respective restriction enzymes (including BglII, NdeI, SacI, AvrII, NotI, BamHI, and SalI). Therefore, a puc-d-loxpKanMX-ENO1-psXDH recombinant vector (6676 bp) having a structure shown in FIG. 5 was obtained.

### Example 3

### Construction of pFENC-Cre Recombinant Vector Containing Cre Recombinase Gene

In this example, a pFENC-Cre recombinant vector containing a Cre recombinase gene was constructed substantially according to the method set forth in Ulrich Güldener et al. (1996), Nucleic Acids Research, 24:2519-2524.

### A. Gene Synthesis of Optimized Cre Recombinase Gene

In order to prepare an optimized Cre recombinase gene that can be expressed in Saccharomyces cerevisiae, the Cre recombinase gene of Enterobacteria phage P1 (NCBI Accession No. YP_006472.1) was subjected to base optimization. Consequently, an optimized Cre recombinase gene, which has a nucleotide sequence of SEQ ID NO:25 (1058 bp), was obtained. Subsequently, the forty-six primers shown in SEQ ID NOs:26 to 71, which were designed using the publicly available computer program DNAWorks, were used to synthesize the optimized Cre recombinase gene.

The gene synthesis of the optimized Cre recombinase gene was completed after the following two PCR processes were conducted. First of all, a solution of a template mixture containing the primers shown in SEQ ID NOs:26 to 71 (each primer at a concentration of 2 μM) was used to conduct a first PCR process according to the method described in section 4 of “General Experimental Procedures”, so that a first PCR product was obtained. Subsequently, a second PCR process was performed with the first PCR product serving as a template and the two primers shown in SEQ ID NOs:26 and 71 according to the method described in section 4 of “General Experimental Procedures”. To determine whether PCR product B1 having a size of about 1058 bp (i.e. the optimized Cre recombinase gene) was obtained, 1% agarose gel electrophoresis was performed after the second PCR process was accomplished.

### B. Cloning of Hygromycin Resistance Gene, GAL1 Promoter, KanMX Fragment and 2u Ori Fragment

First, a PCR process was conducted with a pFA6-hphMX6 plasmid, a pYD1 plasmid, a pFA6a-link-yEGFP-Kan vector and a pSos plasmid (each of which serves as a template) and the four primer pairs shown in Table 3 according to the method described in section 4 of “General Experimental Procedures”. Consequently, a PCR product containing a hygromycin resistance gene (hereinafter referred to as “PCR product B2”; 1674 bp), a PCR product containing a GAL1 promoter (hereinafter referred to as “PCR product B3”; 475 bp), a PCR product containing a KanMX fragment (hereinafter referred to as “PCR product B4”; 1476 bp), and a PCR product containing a 2u ori fragment (hereinafter referred to as “PCR product B5”; 874 bp) were prepared.

TABLE 3

| | | | Size of |
|---|---|---|---|
| | | | PCR |
| | Nucleotide sequence | PCR | product |
| Primer | (5′→3′) | template | (bp) |
| Forward | BamHI | pFA6- | 1674 |
| primer | ggatccggatcctctgttt | hphMX6 | |
| Hyg-F | agcttgcctcg | plasmid | |
| | (SEQ ID NO: 72) | | |
| Reverse | SalI | | |
| primer | gtcgacgtcgacactggat | | |
| Hyg-R | ggcggcgttagtat | | |
| | (SEQ ID NO: 73) | | |
| Forward | SacI | pYD1 | 475 |
| primer | gagctcgagctcacggatt | plasmid | |
| Galp-F | agaagccgccgag | | |
| | (SEQ ID NO: 74) | | |
| Reverse | AvrII | | |
| primer | cctaggcctaggggttttt | | |
| Galp-R | tctccttgacgttaaa | | |
| | (SEQ ID NO: 75) | | |
| Forward | NdeI | pFA6a- | 1476 |
| primer | ttcgcacatatgttcgaga | link- | |
| KMX-NdeI-F | actgctctgtttag | yEGFP- | |
| | (SEQ ID NO: 76) | Kan | |
| Reverse | SacI | vector | |
| primer | atccgtgagctcgttttcg | | |
| KMX-SacI-R | acactggatggcggcgt | | |
| | (SEQ ID NO: 77) | | |
| Forward | BglII | pSos | 874 |
| primer | gtacatagatctgcccctg | plasmid | |
| 2u-F | tgtgttctcgttatgttg | | |
| | (SEQ ID NO: 78) | | |
| Reverse | NdeI | | |
| primer | gtaatccatatgaatattg | | |
| 2u-R | cgaataccgcttccacaa | | |
| | (SEQ ID NO: 79) | | |

Note:
The underlined nucleotides represent the recognition site of the restriction enzyme indicated thereabove.

C. Construction of pFENC-Cre Recombinant Vector

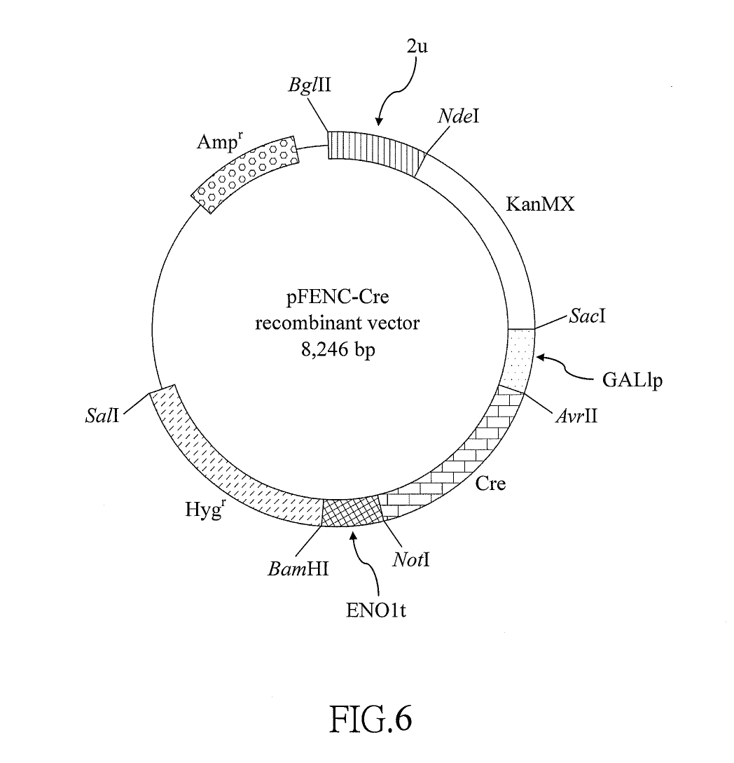

PCR products B1 to B5 obtained from sections A and B of this example and PCR product A5 obtained from section A of Example 2 were all cloned into a pUC19 vector (2686 bp) using respective restriction enzymes (including BglII, NdeI, SacI, AvrII, NotI, BamHI and SalI). Therefore, a pFENC-Cre recombinant vector (8246 bp) having a structure shown in FIG. 6 was constructed.

### Example 4

### Preparation of Saccharomyces cerevisiae Transformants

In order to understand the effects of various gene modifications on the growth and ethanol yield of Saccharomyces cerevisiae DSM 25508, the inventors used the dF fragment from the yTA-FPS-loxpKanMX recombinant vector, the dG1 fragment from the yTA-GPD1-loxpKanMX recombinant vector, the dG2 fragment from the yTA-GPD2-loxpKanMX recombinant vector, the puc-d-loxpKanMX-ENO1-psXDH recombinant vector, and the pFENC-Cre recombinant vector to transform Saccharomyces cerevisiae DSM 25508.

Experimental Procedures:

A. Transformation of Saccharomyces cerevisiae DSM 25508 with dF Fragment, dG1 Fragment, dG2 Fragment or Puc-d-loxpKanMX-ENO1-psXDH Recombinant Vector

The yTA-FPS-loxpKanMX, yTA-GPD1-loxpKanMX and yTA-GPD2-loxpKanMX recombinant vectors constructed in Example 1 served as templates, and the following primers shown in Table 1 were used: FPS1-F-BglII-F (SEQ ID NO:1) and FPS1-R-SalI-R (SEQ ID NO:4) serving as a pair, GPD1-F-XhoI-F (SEQ ID NO:5) and GPD1-R-SalI-R (SEQ ID NO:8) serving as a pair, and GPD2-F-BglII-F (SEQ ID NO:9) and GPD2-R-SalI-R (SEQ ID NO:12) serving as a pair. A PCR process was conducted according to the method set forth in section 4 of “General Experimental Procedures” so as to obtain a dF fragment, a dG1 fragment and a dG2 fragment. In addition, a restriction enzyme XhoI was used to digest the puc-d-loxpKanMX-ENO1-psXDH recombinant vector constructed in Example 2 so as to obtain a linearized puc-d-loxpKanMX-ENO1-psXDH recombinant vector.

Ten groups of cells of Saccharomyces cerevisiae DSM 25508 to be transformed (i.e. ten experimental groups referred to as Experimental Groups 1 to 10) were provided. Subsequently, the Saccharomyces cerevisiae cells in each group were subjected to transformation using at least one of the aforesaid DNA fragments (including a dF fragment, a dG1 fragment, a dG2 fragment, and a linearized puc-d-loxpKanMX-ENO1-psXDH recombinant vector) according to the method set forth in section 5 of “General Experimental Procedures” and the respective transformation step(s) shown in Table 4, so that the resultant Saccharomyces cerevisiae transformants in all the groups carry a desired DNA fragment(s).

During the process of preparing the Saccharomyces cerevisiae transformants, the treatment set forth in section 6 of “General Experimental Procedures” was performed after transformation with the dF fragment, the dG1 fragment and the dG2 fragment. Furthermore, after transformation with the linearized puc-d-loxpKanMX-ENO1-psXDH recombinant vector, a PCR process was conducted to confirm whether the linearized puc-d-loxpKanMX-ENO1-psXDH recombinant vector had been incorporated into the genomic DNA of the Saccharomyces cerevisiae transformants. In addition, the KanMX resistance gene of each Saccharomyces cerevisiae transformant was removed according to the method set forth in section 7 of “General Experimental Procedures” after transformation (either for knockout of a target gene or for overexpression of XDH). Lastly, the Saccharomyces cerevisiae transformants were named according to the respective transformation step(s).

TABLE 4

| | | Transformation |
|---|---|---|
| Group | Transformant | step(s) |
| Experimental Group 1 | 5dF | (1) dF fragment |
| Experimental Group 2 | 5dFdG2 | (1) dF fragment |
| | | (2) dG2 fragment |
| Experimental Group 3 | 5dFdG2XDH | (1) dF fragment |
| | | (2) dG2 fragment |
| | | (3) linearized puc- |
| | | d-loxpKan-MX- |
| | | ENO1-psXDH |
| | | recombinant |
| | | vector |
| Experimental Group 4 | 5dFdG2dG1 | (1) dF fragment |
| | | (2) dG2 fragment |
| | | (3) dG1 fragment |
| Experimental Group 5 | 5dFdG2XDHdG1 | (1) dF fragment |
| | | (2) dG2 fragment |
| | | (3) linearized puc- |
| | | d-loxpKan-MX- |
| | | ENO1-psXDH |
| | | recombinant |
| | | vector |
| | | (4) dG1 fragment |
| Experimental Group 6 | 5dFXDH | (1) dF fragment |
| | | (2) linearized puc- |
| | | d-loxpKan-MX- |
| | | ENO1-psXDH |
| | | recombinant |
| | | vector |
| Experimental Group 7 | 5dFXDHdG1 | (1) dF fragment |
| | | (2) linearized puc- |
| | | d-loxpKan-MX- |
| | | ENO1-psXDH |
| | | recombinant |
| | | vector |
| | | (3) dG1 fragment |
| Experimental Group 8 | 5dFXDHdG2 | (1) dF fragment |
| | | (2) linearized puc- |
| | | d-loxpKan-MX- |
| | | ENO1-psXDH |
| | | recombinant |
| | | vector |
| | | (3) dG2 fragment |
| Experimental Group 9 | 5dFXDHdG1dG2 | (1) dF fragment |
| | | (2) linearized puc- |
| | | d-loxpKan-MX- |
| | | ENO1-psXDH |
| | | recombinant |
| | | vector |
| | | (3) dG1 fragment |
| | | (4) dG2 fragment |
| Experimental Group 10 | 5dFXDHdG2dG1 | (1) dF fragment |
| | | (2) linearized puc- |
| | | d-loxpKan-MX- |
| | | ENO1-psXDH |
| | | recombinant |
| | | vector |
| | | (3) dG2 fragment |
| | | (4) dG1 fragment |

After the transformation, the Saccharomyces cerevisiae transformants in each group were inoculated into a YPD solid medium and were cultivated in a shaking incubator (30° C., 200 rpm) for 3 to 5 days, followed by observing the growth condition.

Result:

The growth condition of the Saccharomyces cerevisiae transformants in each group is shown in Table 5.

TABLE 5

| | | Growth |
|---|---|---|
| Group | Transformant | condition |
| Experimental Group 1 | 5dF | Viable |
| Experimental Group 2 | 5dFdG2 | Viable |
| Experimental Group 3 | 5dFdG2XDH | Viable |
| Experimental Group 4 | 5dFdG2dG1 | Inviable |
| Experimental Group 5 | 5dFdG2XDHdG1 | Inviable |
| Experimental Group 6 | 5dFXDH | Viable |
| Experimental Group 7 | 5dFXDHdG1 | Viable |
| Experimental Group 8 | 5dFXDHdG2 | Viable |
| Experimental Group 9 | 5dFXDHdG1dG2 | Viable |
| Experimental Group 10 | 5dFXDHdG2dG1 | Inviable |

As shown in Table 5, The Saccharomyces cerevisiae transformants in Experimental Groups 1 to 3 and 6 to 9 (i.e. Saccharomyces cerevisiae transformants 5dF, 5dFdG2, 5dFdG2XDH, 5dFXDH, 5dFXDHdG1, 5dFXDHdG2 and 5dFXDHdG1dG2) have a good growth condition, while the Saccharomyces cerevisiae transformants in Experimental Groups 4, 5 and 10 (i.e. Saccharomyces cerevisiae transformants 5dFdG2dG1, 5dFdG2XDHdG1, and 5dFXDHdG2dG1) are inviable. The aforesaid results reveal that knocking out both gpd2 gene and gpd1 gene in a sequential order causes the Saccharomyces cerevisiae transformants to fail to survive, no matter whether the linearized puc-d-loxpKanMX-ENO1-psXDH recombinant vector is incorporated. Consequently, the inventors deduced that the order of knocking out both gpd1 gene and gpd2 gene is important for the growth of the Saccharomyces cerevisiae transformants.

### Example 5

### Evaluation for Xylitol, Glycerol and Ethanol Yields Regarding Various Saccharomyces cerevisiae Transformants

In order to search for a Saccharomyces cerevisiae transformant that does not result in a large amount of undesired by-products (i.e. xylitol and glycerol) and that can efficiently produce ethanol, the inventors used the Saccharomyces cerevisiae transformants in Experimental Groups 1 to 3 and 6 to 9 as obtained in Example 4 for the following experiments.

A. Preparation of Saccharomyces cerevisiae Inoculums

A single colony of cells of Saccharomyces cerevisiae DSM 25508 and a single colony of the Saccharomyces cerevisiae transformants in each of Experimental Groups 1 to 3 and 6 to 9 were each inoculated into a 50 mL test tube containing 10 mL of a YPD60 medium (supplemented with 1% yeast extract, 2% peptone and 6% glucose), followed by cultivation at 30° C. and 150-200 rpm for 24 hours. 4 mL of the resultant yeast solution was inoculated into a 500 mL baffled flask containing 100 mL of a seed medium (supplemented with a 6% (w/v) corn steep liquor and 3% (w/v) cane molasses), followed by cultivation at 30° C. and 150-200 rpm for 16 to 20 hours. Subsequently, centrifugation was performed at 5000 g for 10 minutes, and the supernatant thus formed was removed. The resultant yeast cells were used as a Saccharomyces cerevisiae inoculum in the following experiments.

B. Evaluation for Xylitol, Glycerol and Ethanol Yields Regarding Saccharomyces cerevisiae Transformants 5dF, 5dFdG2, 5dFdG2XDH, 5dFXDH, 5dFXDHdG1, 5dFXDHdG2 and 5dFXDHdG1dG2

The inoculums of the Saccharomyces cerevisiae transformants in Experimental Groups 1 to 3 and 6 to 9 as obtained in section A of this example were used in this section. In addition, the inoculum of Saccharomyces cerevisiae DSM 25508 served as a control group (hereinafter referred to as “Control Group 1”). A suitable amount of the inoculum in each group was added into a 500 mL flask containing 100 mL of a mixed sugar liquor (containing 7% (w/v) of glucose, 4% (w/v) of xylose and 0.1% (w/v) of urea), followed by adjusting the pH value of the resultant mixture to 5.0 with 6N NaOH. Subsequently, a HPLC analysis was performed according to the method set forth in section 8 of “General Experimental Procedures” so as to determine the contents (g/L) of glucose and xylose in the mixture. Fermentation was conducted under an anaerobic condition in a shaking incubator (30° C., 200 rpm) for 72 hours.

The resultant fermented culture was subjected to centrifugation, and the supernatant thus formed was collected, followed by conducting a HPLC analysis according to the method set forth in section 8 of “General Experimental Procedures” so as to determine the contents (g/L) of xylitol, glycerol and ethanol in the fermented culture.

The xylitol yield (g/g) of each group was calculated using the following formula (I):

A=B/C (I)

A: xylitol yield (g/g)

B: xylitol content after fermentation (g/L)

C: xylose content before fermentation (g/L)

The glycerol yield (g/g) of each group was calculated using the following formula (II):

D=E/(F+G) (II)

D: glycerol yield (g/g)

E: glycerol content after fermentation (g/L)

F: glucose content before fermentation (g/L)

G: xylose content before fermentation (g/L)

The ethanol yield (g/g) of each group was calculated using the following formula (III):

H=F(J×0.51+K×0.48) (III)

H: ethanol yield (g/g)

I: ethanol content after fermentation (g/L)

J: glucose content before fermentation (g/L)

K: xylose content before fermentation (g/L)

(Note: The theoretical ethanol conversion rate of glucose is 0.51 g ethanol/g glucose, and the theoretical ethanol conversion rate of xylose is 0.48 g ethanol/g xylose.)

The glucose and xylose contents (g/L) of the mixture in each group and the xylitol, glycerol and ethanol contents (g/L) of the fermented culture in each group are shown in Table 6. The xylitol, glycerol and ethanol yields (g/g) of each group are shown in Table 7.

TABLE 6

| | Mixture | | Fermented culture | | |
|---|---|---|---|---|---|
| | Glucose | Xylose | Xylitol | Glycerol | Ethanol |
| | content | content | content | content | content |
| Group | (g/L) | (g/L) | (g/L) | (g/L) | (g/L) |
| Experimental | 75.04 | 42.56 | 4.16 | 4.94 | 45.65 |
| Group 1 | | | | | |
| Experimental | 75.50 | 42.72 | 4.33 | 4.55 | 47.50 |
| Group 2 | | | | | |
| Experimental | 75.50 | 42.72 | 2.47 | 5.11 | 47.98 |
| Group 3 | | | | | |
| Experimental | 76.19 | 43.23 | 2.25 | 6.88 | 46.41 |
| Group 6 | | | | | |
| Experimental | 75.44 | 42.55 | 2.78 | 5.44 | 46.74 |
| Group 7 | | | | | |
| Experimental | 75.23 | 42.62 | 3.15 | 3.83 | 47.29 |
| Group 8 | | | | | |
| Experimental | 75.61 | 42.63 | 3.47 | 0.29 | 49.18 |
| Group 9 | | | | | |
| Control Group 1 | 75.50 | 42.72 | 7.11 | 6.96 | 42.73 |

TABLE 7

| | Xylitol | Glycerol | Ethanol |
|---|---|---|---|
| | yield | yield | yield |
| Group | (g/g) | (g/g) | (g/g) |
| Experimental | 0.098 | 0.042 | 0.778 |
| Group 1 | | | |
| Experimental | 0.101 | 0.039 | 0.805 |
| Group 2 | | | |
| Experimental | 0.058 | 0.043 | 0.813 |
| Group 3 | | | |
| Experimental | 0.052 | 0.058 | 0.779 |
| Group 6 | | | |
| Experimental | 0.065 | 0.046 | 0.794 |
| Group 7 | | | |
| Experimental | 0.074 | 0.033 | 0.804 |
| Group 8 | | | |
| Experimental | 0.081 | 0.002 | 0.833 |
| Group 9 | | | |
| Control Group 1 | 0.166 | 0.059 | 0.724 |

As shown in Table 7, the xylitol yield of each of Experimental Groups 1 to 3 and 6 to 9 is significantly lower than that of Control Group 1, and Experimental Group 6 has the lowest xylitol yield. Furthermore, the glycerol yield of each of Experimental Groups 1 to 3 and 6 to 9 is lower than that of Control Group 1, and Experimental Group 9 has the lowest glycerol yield. In addition, the ethanol yield of each of Experimental Groups 1 to 3 and 6 to 9 is significantly higher than that of Control Group 1, and Experimental Group 9 has the highest ethanol yield.

The aforesaid results reveal that the Saccharomyces cerevisiae transformants in Experimental Groups 1 to 3 and 6 to 9 can efficiently produce ethanol and only lead to a satisfactorily low amount of undesired by-products (i.e. xylitol and glycerol). Particularly, Saccharomyces cerevisiae transformant 5dFXDHdG1dG2 (i.e. Experimental Group 9) exhibits the highest ethanol productivity and only leads to a significantly low amount of glycerol. In view of the foregoing, a Saccharomyces cerevisiae transformant, which can efficiently produce ethanol and only leads to a satisfactorily low amount of xylitol and glycerol in a fermentation process, can be prepared by sequentially knocking out fps1 gene, overexpressing psXDH gene, and knocking out gpd1 gene and gpd2 gene in Saccharomyces cerevisiae.

C. Evaluation for Xylitol, Glycerol and Ethanol Yields Regarding Various Isolates of Saccharomyces cerevisiae Transformant 5dFXDHdG1dG2

5 single colonies, respectively named 5dFXDHdG1dG2-1, 5dFXDHdG1dG2-2, 5dFXDHdG1dG2-3, 5dFXDHdG1dG2-4 and 5dFXDHdG1dG2-5, were obtained from the culture of Saccharomyces cerevisiae transformant 5dFXDHdG1dG2 (i.e. Experimental Group 9) prepared in Example 4, and were used to prepare inoculums according to the method set forth in section A of this example. Five inoculums of the isolates (hereinafter referred to as Experimental Groups 11 to 15) were obtained. In addition, the inoculum of Saccharomyces cerevisiae DSM 25508 was used as a control group (hereinafter referred to as “Control Group 2”).

Fermentation of the inoculums and determination of the xylitol, glycerol and ethanol yields were conducted according to the method set forth in section B of this example. The results are shown in Table 8.

TABLE 8

| | Xylitol | Glycerol | Ethanol |
|---|---|---|---|
| | yield | yield | yield |
| Group | (g/g) | (g/g) | (g/g) |
| Experimental | 0.068 | 0.003 | 0.797 |
| Group 11 | | | |
| Experimental | 0.088 | 0.0027 | 0.784 |
| Group 12 | | | |
| Experimental | 0.093 | 0.0029 | 0.788 |
| Group 13 | | | |
| Experimental | 0.07 | 0.0032 | 0.807 |
| Group 14 | | | |
| Experimental | 0.071 | 0.0032 | 0.814 |
| Group 15 | | | |
| Control Group 2 | 0.289 | 0.045 | 0.707 |

As shown in Table 8, the xylitol yield and glycerol yield of each of Experimental Groups 11 to 15 are significantly lower than those of Control Group 2, while the ethanol yield of each of Experimental Groups 11 to 15 is significantly higher than that of Control Group 2.

The aforesaid results reveal that various isolates of Saccharomyces cerevisiae transformant 5dFXDHdG1dG2 of the present disclosure can efficiently produce ethanol and only lead to a satisfactorily low amount of undesired by-products (i.e. glycerol and xylitol).

In view of the foregoing, Saccharomyces cerevisiae transformant 5dFXDHdG1dG2-5 (named as “Saccharomyces cerevisiae Sc 206dG2”) has been deposited in the Biosource Collection and Research Center (BCRC) of Food Industry Research and Development Institute (FIRDI) under an accession number BCRC 920086 since Nov. 12, 2013, and has been deposited in the Deutsche Sammlung von Mikroorganismen and Zellkulturen GmbH (DSMZ) under an accession number DSM 28105 since Nov. 28, 2013.

### Example 6

### Xylitol, Glycerol and Ethanol Yields of Saccharomyces cerevisiae Transformant DSM 28105 in Cellulosic Hydrolysate of Rice Straw

In this example, the inventors used a cellulosic hydrolysate of rice straw (which was prepared using a dilute acid as a catalyst via steam explosion according to the method described in Keikhosro Karimi et al. (2006), Biomass and Bioenergy, 30:247-253) as a substrate so as to investigate the xylitol, glycerol and ethanol yields of Saccharomyces cerevisiae transformant DSM 28105.

Experimental Procedure:

The inoculum of Saccharomyces cerevisiae transformant DSM 28105 was used as an experimental group (hereinafter referred to as “Experimental Group 16”), and the inoculum of Saccharomyces cerevisiae DSM 25508 was used as a control group (hereinafter referred to as “Control Group 3”). Substantially according to the method set forth in section B of Example 5, the inoculum in each group was cultivated under an anaerobic condition, and the xylitol, glycerol and ethanol yields of each group were determined. However, a cellulosic hydrolysate of rice straw (100 mL) containing 7% (w/v) of glucose, 4% (w/v) of xylose and 0.1% (w/v) of urea was used instead of 100 mL of the mixed sugar liquor, and the pH value of the resultant mixture was adjusted to 5.2 with 6N NaOH.

Result:

The results are shown in Table 9.

TABLE 9

| | Xylitol | Glycerol | Ethanol |
|---|---|---|---|
| | yield | yield | yield |
| Group | (g/g) | (g/g) | (g/g) |
| Experimental | 0.07 | 0.003 | 0.909 |
| Group 16 | | | |
| Control Group 3 | 0.141 | 0.054 | 0.794 |

As shown in Table 9, when the cellulosic hydrolysate of rice straw is used as a substrate, Saccharomyces cerevisiae transformant DSM 28105 of the present disclosure can efficiently produce ethanol and only leads to a satisfactorily low amount of xylitol and glycerol.

All the patents and references cited in this specification are incorporated herein in their entirety as reference. When there is conflict, the detailed descriptions in this case, including the definitions, would prevail.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

1. A process for producing a recombinant yeast cell, comprising:
providing a parent yeast cell having a genome that includes genes enabling the parent yeast cell to produce ethanol by consuming a hexose and a pentose, a gene encoding xylose reductase (XR), a first gene encoding xylitol dehydrogenase (XDH), and a gene encoding xylulose kinase (XK); and
subjecting the parent yeast cell to a genetic modification treatment, which includes deleting, disrupting, or disabling the fps1 gene in the genome of the parent yeast cell, introducing a second gene encoding XDH into the genome of the parent yeast cell for over-production of XDH, and deleting, disrupting, or disabling the gpd1 and gpd2 genes in the genome of the parent yeast cell in a sequential order.

2. The process of claim 1, wherein the genetic modification treatment includes the sequential steps of:
deleting, disrupting, or disabling the fps1 gene in the genome of the parent yeast cell;
introducing the second gene encoding XDH into the genome of the parent yeast cell for over-production of XDH;
deleting, disrupting, or disabling the gpd1 gene in the genome of the parent yeast cell; and
deleting, disrupting, or disabling the gpd2 gene in the genome of the parent yeast cell.

3. The process of claim 1, wherein the gene encoding XR and the first gene encoding XDH in the genome of the parent yeast cell are exogenous, and are derived from the genome of Pichia stipitis.

4. The process of claim 1, wherein the parent yeast cell is a cell of the Saccharomyces cerevisiae strain deposited under the accession number DSM 25508.

5. The process of claim 1, wherein the recombinant yeast cell is a cell of the yeast strain deposited under the accession number DSM 28105.

6. A recombinant yeast cell, which is produced by a process according to claim 1.

7. The recombinant yeast cell of claim 6, which is a cell of the yeast strain deposited under the accession number DSM 28105.

8. A method for producing ethanol from a biomass containing a hexose and/or a pentose, comprising:
subjecting the biomass to fermentation with a recombinant yeast cell able to produce ethanol by consuming a hexose and a pentose,
wherein the recombinant yeast cell is produced by a process according to claim 1.

9. The method of claim 8, wherein the biomass is a cellulosic hydrolysate of rice straw.


SEQUENCE LISTING (79 sequences)

SEQ ID NO: 1 - DNA, 35 bp, Artificial Sequence
  forward primer FPS1-F-BglII-F for amplifying Fps1-F fragment
agactcagatctctcgtcctaaagtcaattagttc

SEQ ID NO: 2 - DNA, 45 bp, Artificial Sequence
  reverse primer FPS1-F-LoKa-NdeI-R for amplifying Fps1-F fragment
cattatacgaagttatcatatggccttctcactatgagatctgat

SEQ ID NO: 3 - DNA, 41 bp, Artificial Sequence
  forward primer FPS1-R-LoKa-SacI-F for amplifying Fps1-R fragment
gctatacgaagttatgagctctatatatatttacatagatg

SEQ ID NO: 4 - DNA, 34 bp, Artificial Sequence
  reverse primer FPS1-R-SalI-R for ampl ifying Fps1-R fragment
gtcgacgtcgactatagtaggtgaccaggctgag

SEQ ID NO: 5 - DNA, 35 bp, Artificial Sequence
  forward primer GPD1-F-XhoI-F for amplifying Gpd1-F fragment
ctcgagctcgagctaccatgagtgaaactgttacg

SEQ ID NO: 6 - DNA, 46 bp, Artificial Sequence
  reverse primer GPD1-F-LoKa-NdeI-R for amplifying Gpd1-F fragment
cattatacgaagttatcatatgcagaagaggaacttctctttctac

SEQ ID NO: 7 - DNA, 46 bp, Artificial Sequence
  forward primer GPD1-R-LoKa-SacI-F for amplifying Gpd1-R fragment
gctatacgaagttatgagctcgttcacgaatggttggaaacatgtg

SEQ ID NO: 8 - DNA, 37 bp, Artificial Sequence
  reverse primer GPD1-R-SalI-R for amplifying Gpd1-R fragment
gtcgacgtcgacgacataatgctaatttatgaatatg

SEQ ID NO: 9 - DNA, 35 bp, Artificial Sequence
  forward primer GPD2-F-BglII-F for amplifying Gpd2-F fragment
agactcagatctgagtatgctcgaaacaataagac

SEQ ID NO: 10 - DNA, 44 bp, Artificial Sequence
  reverse primer GPD2-F-LoKa-NdeI-R for amplifying Gpd2-F fragment
cattatacgaagttatcatatggagaagagctgctgaacaatag

SEQ ID NO: 11 - DNA, 43 bp, Artificial Sequence
  forward primer GPD2-R-LoKa-SacI-F for amplifying Gpd2-R fragment
gctatacgaagttatgagctccattatacacaagttctacaac

SEQ ID NO: 12 - DNA, 36 bp, Artificial Sequence
  reverse primer GPD2-R-SalI-R for amplifying Gpd2-R fragment
gtcgacgtcgaccgataatagcgtgtataatggtag

SEQ ID NO: 13 - DNA, 76 bp, Artificial Sequence
  forward primer loxpKanMX-NdeI-F for amplifying Loxp-KanMX-Loxp fragment
catatgcatatgataacttcgtataatgtatgctatacgaagttatttcgagaactgctctgtttagcttgcctcg

SEQ ID NO: 14 - DNA, 76 bp, Artificial Sequence
  reverse primer loxpKanMX-SacI-R for amplifying Loxp-KanMX-Loxp fragment
gagttcgagctcataacttcgtatagcatacattatacgaagttatgttttcgacactggatggcggcgttagtat

SEQ ID NO: 15 - DNA, 36 bp, Artificial Sequence
  forward primer psXDH-AvrII-F for amplifying psXDH gene
cctaggcctaggatgactgctaacccttccttggtg

SEQ ID NO: 16 - DNA, 35 bp, Artificial Sequence
  reverse primer psXDH-NotI-R for amplifying psXDH gene
gcggccgcggccgcttactcagggccgtcaatgag

SEQ ID NO: 17 - DNA, 28 bp, Artificial Sequence
  forward primer Delta-BglII-F for amplifying Delta sequence
ctgtcaagatcttgttggaatagaaatc

SEQ ID NO: 18 - DNA, 30 bp, Artificial Sequence
  reverse primer Delta-NdeI-R for amplifying Delta sequence
ctgtcacatatgaaatgggtgaatgttgag

SEQ ID NO: 19 - DNA, 28 bp, Artificial Sequence
  forward primer Delta-BamHI-F for amplifying Delta sequence
ctgtcaggatcctgttggaatagaaatc

SEQ ID NO: 20 - DNA, 30 bp, Artificial Sequence
  reverse primer Delta-SalI-R for amplifying Delta sequence
ctgtcagtcgacaaatgggtgaatgttgag

SEQ ID NO: 21 - DNA, 30 bp, Artificial Sequence
  forward primer ENO1p-SacI-F for amplifying ENO1 promoter
gagctcgagctccggaaccgccagatattc

SEQ ID NO: 22 - DNA, 35 bp, Artificial Sequence
  reverse primer ENO1p-AvrII-R for amplifying ENO1 promoter
cctaggcctaggtttgatttagtgtttgtgtgttg

SEQ ID NO: 23 - DNA, 42 bp, Artificial Sequence
  forward primer ENO1t-NotI-F for amplifying ENO1 terminator
gcggccgcggccgcgaattcagtttttgattaagccttctag

SEQ ID NO: 24 - DNA, 37 bp, Artificial Sequence
  reverse primer ENO1t-BamHI-R for amplifying ENO1 terminator
ggatccggatcccttcattgagcttagaacccttttg

SEQ ID NO: 25 - DNA, 1058 bp, Artificial Sequence
  the optimized Cre recombinase gene
cctaggcctaggatgtctaatttattaaccgtacaccaaaatttgccagcattgccggtcgatgcaacttctgatgaggttagaaagaacttaatggacatgttcagggatagacaggctttttctgagcatacctggaaaatgttgttgtctgtttgcagatcatgggctgcatggtgcaagttgaataacagaaaatggtttccagcagaacctgaagatgttagagattatttgttgtatttgcaggctagaggtttggcagtaaaaactatccagcaacatttgggtcagttgaacatgttgcatagaagatcaggtttgccaagaccatctgactctaatgctgtttcattggttatgagaagaatcagaaaagaaaacgttgatgctggtgaaagagcaaaacaggctttggcttttgaaagaactgatttcgaccaggttagatcattgatggaaaattctgatagatgccaggatataagaaatttggcatttttgggtattgcttataacaccttgttaagaatagccgaaattgccaggatcagggttaaagatatttcaagaactgacggtggtagaatgttaatccatattggtagaactaaaactttggtttctaccgcaggtgtagagaaggcattgtctttgggtgtaactaaattggtcgagagatggattttggtctctggtgtagctgatgatccaaataactacttgttttgcagagtcagaaaaaatggtgttgccgcaccatctgccacctctcagttgtcaactagagccttggaaggtatttttgaagcaactcatagattgatttacggtgctaaggatgactctggtcagagatacttggcctggtctggacactctgccagagtcggagccgcaagagatatggctagagctggagtttcaataccagagatcatgcaagctggtggttggaccaatgtaaacattgtcatgaactatatcagaaacttggattctgaaacaggtgcaatggtcagattgttggaagatggtgattaggcggccgcgcggcc

SEQ ID NO: 26 - DNA, 38 bp, Artificial Sequence
  primer Cre-1 for the synthesis of the optimized Cre recombinase gene
cctaggcctaggatgtctaatttattaaccgtacacca

SEQ ID NO: 27 - DNA, 44 bp, Artificial Sequence
  primer Cre-2 for the synthesis of the optimized Cre recombinase gene
cggcaatgctggcaaattttggtgtacggttaataaattagaca

SEQ ID NO: 28 - DNA, 41 bp, Artificial Sequence
  primer Cre-3 for the synthesis of the optimized Cre recombinase gene
aaatttgccagcattgccggtcgatgcaacttctgatgagg

SEQ ID NO: 29 - DNA, 47 bp, Artificial Sequence
  primer Cre-4 for the synthesis of the optimized Cre recombinase gene
gaacatgtccattaagttctttctaacctcatcagaagttgcatcga

SEQ ID NO: 30 - DNA, 48 bp, Artificial Sequence
  primer Cre-5 for the synthesis of the optimized Cre recombinase gene
ttagaaagaacttaatggacatgttcagggatagacaggctttttctg

SEQ ID NO: 31 - DNA, 45 bp, Artificial Sequence
  primer Cre-6 for the synthesis of the optimized Cre recombinase gene
caacaacattttccaggtatgctcagaaaaagcctgtctatccct

SEQ ID NO: 32 - DNA, 43 bp, Artificial Sequence
  primer Cre-7 for the synthesis of the optimized Cre recombinase gene
agcatacctggaaaatgttgttgtctgtttgcagatcatgggc

SEQ ID NO: 33 - DNA, 41 bp, Artificial Sequence
  primer Cre-8 for the synthesis of the optimized Cre recombinase gene
ttattcaacttgcaccatgcagcccatgatctgcaaacaga

SEQ ID NO: 34 - DNA, 42 bp, Artificial Sequence
  primer Cre-9 for the synthesis of the optimized Cre recombinase gene
tgcatggtgcaagttgaataacagaaaatggtttccagcaga

SEQ ID NO: 35 - DNA, 47 bp, Artificial Sequence
  primer Cre-10 for the synthesis of the optimized Cre recombinase gene
acaaataatctctaacatcttcaggttctgctggaaaccattttctg

SEQ ID NO: 36 - DNA, 47 bp, Artificial Sequence
  primer Cre-11 for the synthesis of the optimized Cre recombinase gene
acctgaagatgttagagattatttgttgtatttgcaggctagaggtt

SEQ ID NO: 37 - DNA, 43 bp, Artificial Sequence
  primer Cre-12 for the synthesis of the optimized Cre recombinase gene
tgctggatagtttttactgccaaacctctagcctgcaaataca

SEQ ID NO: 38 - DNA, 44 bp, Artificial Sequence
  primer Cre-13 for the synthesis of the optimized Cre recombinase gene
tggcagtaaaaactatccagcaacatttgggtcagttgaacatg

SEQ ID NO: 39 - DNA, 44 bp, Artificial Sequence
  primer Cre-14 for the synthesis of the optimized Cre recombinase gene
gcaaacctgatcttctatgcaacatgttcaactgacccaaatgt

SEQ ID NO: 40 - DNA, 45 bp, Artificial Sequence
  primer Cre-15 for the synthesis of the optimized Cre recombinase gene
ttgcatagaagatcaggtttgccaagaccatctgactctaatgct

SEQ ID NO: 41 - DNA, 48 bp, Artificial Sequence
  primer Cre-16 for the synthesis of the optimized Cre recombinase gene
tgattcttctcataaccaatgaaacagcattagagtcagatggtcttg

SEQ ID NO: 42 - DNA, 47 bp, Artificial Sequence
  primer Cre-17 for the synthesis of the optimized Cre recombinase gene
gtttcattggttatgagaagaatcagaaaagaaaacgttgatgctgg

SEQ ID NO: 43 - DNA, 43 bp, Artificial Sequence
  primer Cre-18 for the synthesis of the optimized Cre recombinase gene
aaagcctgttttgctctttcaccagcatcaacgttttcttttc

SEQ ID NO: 44 - DNA, 44 bp, Artificial Sequence
  primer Cre-19 for the synthesis of the optimized Cre recombinase gene
tgaaagagcaaaacaggctttggcttttgaaagaactgatttcg

SEQ ID NO: 45 - DNA, 46 bp, Artificial Sequence
  primer Cre-20 for the synthesis of the optimized Cre recombinase gene
ttccatcaatgatctaacctggtcgaaatcagttctttcaaaagcc

SEQ ID NO: 46 - DNA, 49 bp, Artificial Sequence
  primer Cre-21 for the synthesis of the optimized Cre recombinase gene
accaggttagatcattgatggaaaattctgatagatgccaggatataag

SEQ ID NO: 47 - DNA, 50 bp, Artificial Sequence
  primer Cre-22 for the synthesis of the optimized Cre recombinase gene
gcaatacccaaaaatgccaaatttcttatatcctggcatctatcagaatt

SEQ ID NO: 48 - DNA, 50 bp, Artificial Sequence
  primer Cre-23 for the synthesis of the optimized Cre recombinase gene
aaatttggcatttttgggtattgcttataacaccttgttaagaatagccg

SEQ ID NO: 49 - DNA, 47 bp, Artificial Sequence
  primer Cre-24 for the synthesis of the optimized Cre recombinase gene
taaccctgatcctggcaatttcggctattcttaacaaggtgttataa

SEQ ID NO: 50 - DNA, 45 bp, Artificial Sequence
  primer Cre-25 for the synthesis of the optimized Cre recombinase gene
aaattgccaggatcagggttaaagatatttcaagaactgacggtg

SEQ ID NO: 51 - DNA, 52 bp, Artificial Sequence
  primer Cre-26 for the synthesis of the optimized Cre recombinase gene
gttctaccaatatggattaacattctaccaccgtcagttcttgaaatatctt

SEQ ID NO: 52 - DNA, 51 bp, Artificial Sequence
  primer Cre-27 for the synthesis of the optimized Cre recombinase gene
gtagaatgttaatccatattggtagaactaaaactttggtttctaccgcag

SEQ ID NO: 53 - DNA, 46 bp, Artificial Sequence
  primer Cre-28 for the synthesis of the optimized Cre recombinase gene
caaagacaatgccttctctacacctgcggtagaaaccaaagtttta

SEQ ID NO: 54 - DNA, 46 bp, Artificial Sequence
  primer Cre-29 for the synthesis of the optimized Cre recombinase gene
gtgtagagaaggcattgtctttgggtgtaactaaattggtcgagag

SEQ ID NO: 55 - DNA, 44 bp, Artificial Sequence
  primer Cre-30 for the synthesis of the optimized Cre recombinase gene
acaccagagaccaaaatccatctctcgaccaatttagttacacc

SEQ ID NO: 56 - DNA, 45 bp, Artificial Sequence
  primer Cre-31 for the synthesis of the optimized Cre recombinase gene
atggattttggtctctggtgtagctgatgatccaaataactactt

SEQ ID NO: 57 - DNA, 48 bp, Artificial Sequence
  primer Cre-32 for the synthesis of the optimized Cre recombinase gene
ccattttttctgactctgcaaaacaagtagttatttggatcatcagct

SEQ ID NO: 58 - DNA, 41 bp, Artificial Sequence
  primer Cre-33 for the synthesis of the optimized Cre recombinase gene
ttttgcagagtcagaaaaaatggtgttgccgcaccatctgc

SEQ ID NO: 59 - DNA, 41 bp, Artificial Sequence
  primer Cre-34 for the synthesis of the optimized Cre recombinase gene
gctctagttgacaactgagaggtggcagatggtgcggcaac

SEQ ID NO: 60 - DNA, 46 bp, Artificial Sequence
  primer Cre-35 for the synthesis of the optimized Cre recombinase gene
cctctcagttgtcaactagagccttggaaggtatttttgaagcaac

SEQ ID NO: 61 - DNA, 49 bp, Artificial Sequence
  primer Cre-36 for the synthesis of the optimized Cre recombinase gene
cttagcaccgtaaatcaatctatgagttgcttcaaaaataccttccaag

SEQ ID NO: 62 - DNA, 48 bp, Artificial Sequence
  primer Cre-37 for the synthesis of the optimized Cre recombinase gene
tcatagattgatttacggtgctaaggatgactctggtcagagatactt

SEQ ID NO: 63 - DNA, 41 bp, Artificial Sequence
  primer Cre-38 for the synthesis of the optimized Cre recombinase gene
gagtgtccagaccaggccaagtatctctgaccagagtcatc

SEQ ID NO: 64 - DNA, 41 bp, Artificial Sequence
  primer Cre-39 for the synthesis of the optimized Cre recombinase gene
ggcctggtctggacactctgccagagtcggagccgcaagag

SEQ ID NO: 65 - DNA, 41 bp, Artificial Sequence
  primer Cre-40 for the synthesis of the optimized Cre recombinase gene
ttgaaactccagctctagccatatctcttgcggctccgact

SEQ ID NO: 66 - DNA, 41 bp, Artificial Sequence
  primer Cre-41 for the synthesis of the optimized Cre recombinase gene
ggctagagctggagtttcaataccagagatcatgcaagctg

SEQ ID NO: 67 - DNA, 42 bp, Artificial Sequence
  primer Cre-42 for the synthesis of the optimized Cre recombinase gene
tgtttacattggtccaaccaccagcttgcatgatctctggta

SEQ ID NO: 68 - DNA, 47 bp, Artificial Sequence
  primer Cre-43 for the synthesis of the optimized Cre recombinase gene
gtggttggaccaatgtaaacattgtcatgaactatatcagaaacttg

SEQ ID NO: 69 - DNA, 48 bp, Artificial Sequence
  primer Cre-44 for the synthesis of the optimized Cre recombinase gene
ccattgcacctgtttcagaatccaagtttctgatatagttcatgacaa

SEQ ID NO: 70 - DNA, 44 bp, Artificial Sequence
  primer Cre-45 for the synthesis of the optimized Cre recombinase gene
gattctgaaacaggtgcaatggtcagattgttggaagatggtga

SEQ ID NO: 71 - DNA, 40 bp, Artificial Sequence
  primer Cre-46 for the synthesis of the optimized Cre recombinase gene
ggccgcgcggccgcctaatcaccatcttccaacaatctga

SEQ ID NO: 72 - DNA, 30 bp, Artificial Sequence
  forward primer Hyg-F for amplifying hygromycin resistance gene
ggatccggatcctctgtttagcttgcctcg

SEQ ID NO: 73 - DNA, 33 bp, Artificial Sequence
  reverse primer Hyg-R for amplifying hygromycin resistance gene
gtcgacgtcgacactggatggcggcgttagtat

SEQ ID NO: 74 - DNA, 32 bp, Artificial Sequence
  forward primer Galp-F for amplifying GALl promoter
gagctcgagctcacggattagaagccgccgag

SEQ ID NO: 75 - DNA, 35 bp, Artificial Sequence
  reverse primer Galp-R for amplifying GALl promoter
cctaggcctaggggttttttctccttgacgttaaa

SEQ ID NO: 76 - DNA, 33 bp, Artificial Sequence
  forward primer KMX-NdeI-F for amplifying KanMX fragment
ttcgcacatatgttcgagaactgctctgtttag

SEQ ID NO: 77 - DNA, 36 bp, Artificial Sequence
  reverse primer KMX-SacI-R for amplifying KanMX fragment
atccgtgagctcgttttcgacactggatggcggcgt

SEQ ID NO: 78 - DNA, 37 bp, Artificial Sequence
  forward primer 2u-F for amplifying 2u ori fragment
gtacatagatctgcccctgtgtgttctcgttatgttg

SEQ ID NO: 79 - DNA, 37 bp, Artificial Sequence
  reverse primer 2u-R for amplifying 2u ori fragment
gtaatccatatgaatattgcgaataccgcttccacaa